(19) United States
(12) Patent Application Publication
(10) Pub. No.: US 2023/0258748 A1
(43) Pub. Date: Aug. 17, 2023

(54) SYSTEM AND APPARATUS FOR OVERLAPPING PHASED-ARRAY COILS FOR CURVED SURFACES

(71) Applicant: NEW YORK SOCIETY FOR THE RELIEF OF THE RUPTURED AND CRIPPLED, MAINTAINING THE HOSPITAL FOR SPECIAL, New York, NY (US)

(72) Inventors: Ek TAN, Westbury, NY (US); Darryl B. Sneag, Plainview, NY (US)

(21) Appl. No.: 18/005,116

(22) PCT Filed: Jul. 26, 2021
(86) PCT No.: PCT/US2021/043204

§ 371 Date: Jan. 11, 2023

Related U.S. Application Data
(60) Provisional application No. 63/058,725, filed on Jul. 30, 2020.

(51) Int. Cl. G01R 33/34

(52) U.S. Cl. CPC G01R 33/34084; A61B 5/055

(57) ABSTRACT

A magnetic resonance (MR) coil assembly includes a first row of coil elements, wherein the first row is a curved row and configured to conform to a first body curvature in a cone shape when the first row is bent and wrapped around the first body curvature; and a second row of coil elements, wherein the second row is a straight row and configured to conform to a second body curvature in a cylinder shape when the second row is bent and wrapped around the second body curvature, wherein each coil element in the first row has a first area and each coil element in the second row has a second area, and wherein the first area is larger than the second area. The first curved row can also be deployed without a second curved row.

### CROSS-REFERENCE TO RELATED APPLICATIONS

This application claims the benefit of priority under 35 U.S.C. § 119 to U.S. Provisional Application Ser. No. 63/058,725, filed Jul. 30, 2020, the entirety of which is incorporated herein by reference.

### TECHNICAL FIELD

This description generally relates to magnetic resonance imaging (MRI).

### BACKGROUND

MRI provides soft-tissue images with superior contrast compared to other imaging modalities and has therefore become widely used for human imaging. Image quality produced by an MRI scanner is affected by the arrangement of, for example, radiofrequency (RF) phased-array receiver coils positioned in the vicinity of the area to be imaged. While the design of these RF coils may factor in competing considerations, concave anatomical surfaces (for example, in the head-neck-shoulder region) present inherent challenges that had not been addressed thus far.

### SUMMARY

In one aspect, some implementations of phased-array receiver RF coils in MRI are coil assemblies that include: a first row of coil elements, wherein the first row is a curved row and configured to conform to a first body curvature in a cone shape when the first row is bent and wrapped around the first body curvature; and a second row of coil elements, wherein the second row is a straight row and configured to conform to a second body curvature in a cylinder shape when the second row is bent and wrapped around the second body curvature, wherein each coil element in the first row has a first area and each coil element in the second row has a second area, and wherein the first area may be equal to, smaller than or larger than the second area.

Implementations may include one or more of the following features:
In some embodiments the first row of coils is curved and exists on its own without a second row.

The coil assembly may additionally include a third row of coil elements, wherein the third row is a curved row and configured to conform to a third body curvature in a cone shape when the third row is bent and wrapped around the third body curvature. The first body curvature may correspond to a juncture between a head region and a neck region. The second body curvature may correspond to the neck region. The third body curvature may correspond to a juncture between the neck region and a shoulder region.

The first body curvature may correspond to a juncture between a groin region and a hip region. The second body curvature may correspond to the groin region. The third body curvature may correspond to a juncture between the groin region and a thigh region.

The first row and the second row may be neighboring rows. Each coil element of the first row may overlap with one or more coil elements of the second row. Each coil element of the first row may overlap with two other coil elements of the first row when the first row is bent and wrapped around the first body curvature. Each coil element of the second row may overlap with one or more coil elements of the first row. Each coil element of the second row may overlap with two other coil elements of the second row when the second row is bent and wrapped around the second body curvature.

The second row and the third row are neighboring rows. Each coil element of the third row may overlap with one or more coil elements of the second row. Each coil element of the third row may overlap with two other coil elements of the third row when the third row is bent and wrapped around the third body curvature. Each coil element of the second row may overlap with one or more coil elements of the third row. Each coil element of the second row overlaps with two other coil elements of the second row when the second row is bent and wrapped around the second body curvature.

Each element in the third row may have a third area. The third area may be larger than the second area. A coil element in the third row may have one of: a circular shape, an elliptical shape, a trapezoidal shape, or a clamshell shape. A coil element in the first row may have one of: a circular shape, an elliptical shape, a trapezoidal shape, or a clamshell shape. A coil element in the second row may have one of: a circular shape, an elliptical shape, or a rectangular shape.

In another aspect, some implementations provide an MRI scanner that include: a main magnet configured to generate a volume of magnetic field with field inhomogeneity below a defined threshold, the main magnet including a bore area sized to accommodate at least a body region of a subject; an RF phased-array receiver coil assembly capable of wrapping around the body region, the coil assembly comprising: a first row of coil elements arranged in a curved row and configured to conform to a first body curvature in a cone shape when the first row is bent and wrapped around the first body curvature; and a second row of coil elements arranged in a straight row and configured to conform to a second body curvature in a cylindrical shape when the second row is bent and wrapped around the second body curvature, wherein each coil element in the first row has a first area and each coil element in the second row has a second area, wherein the first area is larger than the second area, and wherein the first body curvature and the second body curvature are within the body region, gradient coils configured to generate gradient pulses that provide perturbations to the volume of magnetic field such that MRI signals encoding an MRI image according to encoding information from the gradient pulses are emitted from the body region and are subsequently acquired by the coil assembly wrapped around the body region; and a control unit in communication with gradient coils and the coil assembly and configured to operate: (i) the gradient coils to generate the gradient pulses and (ii) the coil assembly to acquire MRI signals emitted from the body region that encode the MRI image.

Implementations may include one or more of the following features:

The MRI scanner may additionally include a third row of coil elements, wherein the third row is a curved row and configured to conform to a third body curvature in a cone shape when the third row is bent and wrapped around the third body curvature. The first body curvature may correspond to a juncture between a head region and a neck region. The second body curvature may correspond to the neck region. The third body curvature may correspond to a juncture between the neck region and a shoulder region.

The first body curvature may correspond to a juncture between a groin region and a hip region. The second body curvature may correspond to the groin region. The third body curvature may correspond to a juncture between the groin region and a thigh region.

The first row and the second row may be neighboring rows. Each coil element of the first row may overlap with one or more coil elements of the second row. Each coil element of the first row may overlap with two other coil elements of the first row when the first row is bent and wrapped around the first body curvature. Each coil element of the second row may overlap with one or more coil elements of the first row. Each coil element of the second row may overlap with two other coil elements of the second row when the second row is bent and wrapped around the second body curvature.

The second row and the third row are neighboring rows. Each coil element of the third row may overlap with one or more coil elements of the second row. Each coil element of the third row may overlap with two other coil elements of the third row when the third row is bent and wrapped around the third body curvature. Each coil element of the second row may overlap with one or more coil elements of the third row. Each coil element of the second row overlaps with two other coil elements of the second row when the second row is bent and wrapped around the second body curvature.

Each element in the third row may have a third area. The third area may be larger than the second area. A coil element in the third row may have one of: a circular shape, an elliptical shape, a trapezoidal shape, or a clamshell shape. A coil element in the first row may have one of: a circular shape, an elliptical shape, a trapezoidal shape, or a clamshell shape. A coil element in the second row may have one of: a circular shape, an elliptical shape, or a rectangular shape.

The details of one or more aspects of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

Embodiments will now be described, by way of example only, with reference to the drawings, in which:

FIG. 1 shows an example of a magnetic resonance imaging (MRI) system for imaging a human.

FIG. 2 illustrates examples of phased-array designs for imaging concave surfaces according to some implementations.

FIG. 3A illustrates examples of 1-D arrays according to some implementations.

FIG. 3B show 3-D views of the example of the 1-D arrays from FIG. 3A.

FIG. 3C shows 3-D views of multiple rows of 1-D arrays according to some implementations.

FIG. 4A to 4B illustrate examples of 2-D arrays with coil elements having non-circular geometries according to some implementations.

FIG. 4C illustrates an example of a clamshell element layout and an array configuration that includes a row of clamshell elements according to some implementations.

Like reference symbols in the various drawings indicate like elements.

### DETAILED DESCRIPTION

A magnetic resonance imaging (MRI) scanner includes one or more radio frequency (RF) coils for receiving magnetic resonance (MR) signals emitted from a sample in response to RF excitation pulses. The advantages of using smaller, multiple RF coils in an array (also known as phased-array coils), as compared to a larger, single coil, include higher signal-to-noise ratio (SNR) and improved capabilities for accelerating MRI acquisitions by means of parallel imaging. Some commercially-available RF receiver coils include multi-element phased-array coils, with the number of elements ranging from, for example, 4 to 128. These coil elements are often rectilinear or circular in shape, and are often arranged to either overlap with one another or with gaps between them (also called “underlap”). One consideration for overlapping or underlapping coils is to provide adequate coverage of the imaged anatomy. Another consideration is the proximity of the coils to the imaged anatomy, as SNR decreases with distance between the two. A third consideration is the size of the coil elements: smaller coil elements increase SNR (up to the point where coil resistance dominates), but at the cost of signal inhomogeneity, increased complexity of manufacturing a high-element count arrays, and increased complexity in reconstructing images from multiple coil sources. Additional considerations include the degree of overlap and intersection angles at which coils overlap, which are important for optimizing coverage and sensitivity to the anatomy and for reducing mutual inductance between the coils. High mutual inductance between coils results in difficulties during tuning of the elements in the coil array, as well as ineffective signal acquisition. Implementations in the present disclosure describe phased-array coils for inherently curved geometries, such as a head-neck-shoulder region that includes a narrower middle section and wider outer sections towards the head and the shoulder, which are different from planar or spherical geometries. In these geometries, configurations using a simple two-dimensional planar array with identical elements will result in large spatial offsets from the concave anatomical surfaces. Indeed, such challenges are inherent for imaging the head/neck/chest wall/shoulder and pelvic/inner thigh regions. Similar challenges also arise when imaging the axillary (armpit) region, the elbow region, the wrist region, fingers, knee, ankle, and toes.

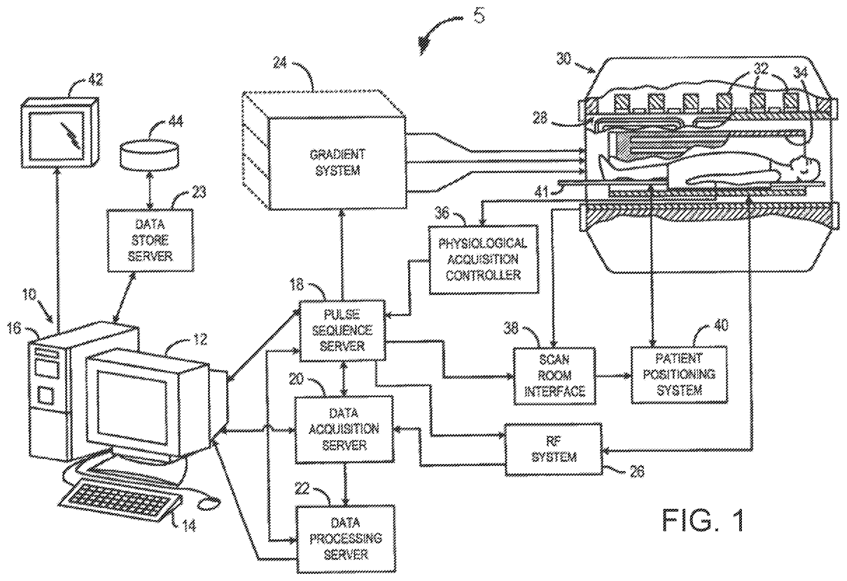

FIG. 1 shows an example of a magnetic resonance imaging (MRI) system 5 with a solenoid magnet for imaging knee joints. The MRI system 5 includes a workstation 10 having a display 12 and a keyboard 14. The Workstation 10 includes a processor 16 that is a commercially available programmable machine running a commercially available operating system. The workstation 10 provides the operator interface that enables scan prescriptions to be entered into the MRI system 5. The workstation 10 is coupled to four servers including a pulse sequence server 18, a data acquisition server 20, a data processing server 22, and a data store server 23. The work station 10 and each server 18, 20, 22 and 23 are connected to communicate with each other.

The pulse sequence server 18 functions in response to instructions downloaded from the workstation 10 to operate a gradient system 24 and an RF system 26. Gradient waveforms necessary to perform the prescribed scan are produced and applied to the gradient system 24 that excites gradient coils in an assembly 28 to produce the magnetic field gradients Gx, Gy and Gz used for position-encoding MR signals. The gradient coil assembly 28 forms part of a magnet assembly 30 that includes a polarizing magnet 32 and a whole-body RF coil 34.

RF excitation waveforms are applied to the RF coil 34 by the RF system 26 to perform the prescribed magnetic resonance pulse sequence. Responsive MR signals detected by the RF coil 34 or a separate local coil (not shown in FIG. 1) are received by the RF system 26, amplified, demodulated, filtered, and digitized under direction of commands produced by the pulse sequence server 18. The RF system 26 includes an RF transmitter for producing a wide variety of RF pulses used in MR pulse sequences. The RF transmitter is responsive to the scan prescription and direction from the pulse sequence server 18 to produce RF pulses of the desired frequency, phase and pulse amplitude waveform. The generated RF pulses may be applied to the whole body RF coil 34 or to one or more local coils or coil arrays (not highlighted in FIG. 1).

The RF system 26 also includes one or more RF receiver channels. Each RF receiver channel includes an RF amplifier that amplifies the MR signal received by the coil to which it is connected and a detector that detects and digitizes the I and Q quadrature components of the received MR signal.

The pulse sequence server 18 also optionally receives patient or subject data from a physiological acquisition controller 36. The controller 36 receives signals from a number of different sensors connected to the patient, such as ECG signals from electrodes or respiratory signals from a bellows. Such signals are typically used by the pulse sequence server 18 to synchronize, or “gate”, the performance of the scan with the subject's respiration or heartbeat.

The pulse sequence server 18 also connects to a scan room interface circuit 38 that receives signals from various sensors associated with the condition of the patient and the magnet system. It is also through the scan room interface circuit 38 that a patient positioning system 40 receives commands to move the patient to desired positions during the scan by translating the patient table 41.

The digitized MR signal samples produced by the RF system 26 are received by the data acquisition server 20. The data acquisition server 20 operates in response to instructions downloaded from the workstation 10 to receive the real-time MR data and provide buffer storage such that no data is lost by data overrun. In some scans the data acquisition server 20 does little more than pass the acquired MR data to the data processor server 22. However, in scans that require information derived from acquired MR data to control the further performance of the scan, the data acquisition server 20 is programmed to produce such information and convey it to the pulse sequence server 18. For example, during prescans, MR data is acquired and used to calibrate the pulse sequence performed by the pulse sequence server 18. Also, navigator signals may be acquired during a scan and used to adjust RF or gradient system operating parameters or to control the view order in which k-space is sampled. In all these examples the data acquisition server 20 acquires MR data and processes it in real-time to produce information that is used to control the scan.

The data processing server 22 receives MR data from the data acquisition server 20 and processes it in accordance with instructions downloaded from the workstation 10. Such processing may include, for example, Fourier transformation of raw k-space MR data to produce two or three dimensional images, the application of filters to a reconstructed image, the performance of a back projection image reconstruction of acquired MR data; the calculation of functional MR images, the calculation of motion or flow images, and the like.

Images reconstructed by the data processing server 22 are conveyed back to the workstation 10 where they are stored. Real-time images are stored in a data base memory cache (not shown) from which they may be output to operator display 12 or a display 42 that is located near the magnet assembly 30 for use by physicians. Batch mode images or selected real time images are stored in a host database on disc storage 44. When such images have been reconstructed and transferred to storage, the data processing server 22 notifies the data store server 23 on the workstation 10. The Workstation 10 may be used by an operator to archive the images, produce films, or send the images via a network to other facilities.

As shown in FIG. 1, the RF system 26 may be connected to the whole body RF coil 34 while a transmitter section of the RF system 26 may connect to one RF coil 152A and its receiver section may connect to a separate RF receive coil 152B. Often, the transmitter section is connected to the whole body RF coil 34 and each receiver section is connected to a separate local coil 152B. In this illustration, RF receive coil 152B can be a phased array coil. In some cases, the phased array coil is a receive-only coil. In other cases, the phased array coil functions as both a transmitter and a receiver (also known as a transceiver).

A phased array coil can improve the SNR of the received MR signals as well as facilitate potential parallel acquisition of the MR signals. A two-dimensional, rectilinear, phased-array can use identical array elements that are overlapped in a rectilinear fashion. The use of identical elements provides manufacturing simplicity, reduced cost, uniform coil penetration depth (i.e., dependent on the coil size), and improved SNR, which are advantageous for imaging. The phased array can be positioned flat in a single plane for imaging, for example, the thoracolumbar spine, e.g., where an individual lies in a supine (flat on back) position. The phased array may also be rolled up in one axis into a cylinder for imaging the arm or leg. Alternatively or additionally, circular elements can be constructed in a spherical “soccer-ball” tessellation for circular anatomies such as the head. In a similar vein, one-dimensional or two-dimensional arrays can be wrapped around like a cylinder to better conform to cylinder-like anatomical geometries. In these examples, the array elements are either identical or very close in size. As discussed earlier, configurations using a simple two-dimensional planar array with identical elements will result in large spatial offsets from the concave anatomical surfaces. Indeed, such challenges are inherent for imaging the head/neck/chest wall/shoulder region, the axillary (armpit) region, the pelvic/inner thigh region, elbow, wrist, fingers, knee, ankle, and toes.

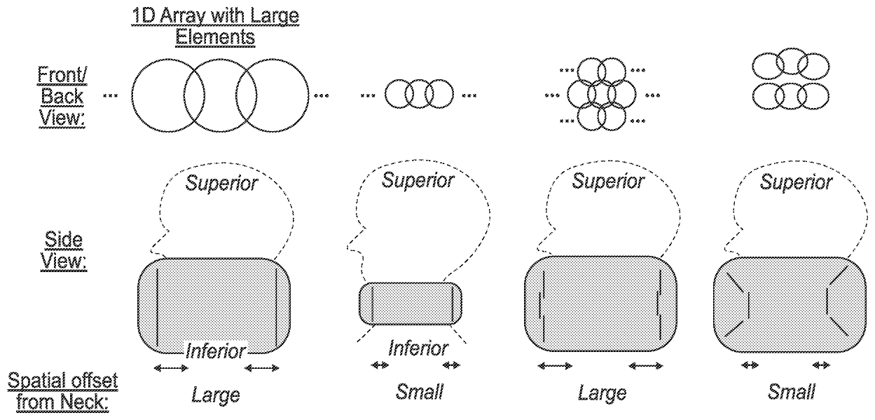

FIG. 2 illustrates examples of phased-array designs for imaging concave surfaces. By comparing and contrasting these examples, the inherent challenges can be further highlighted. In configuration 200, a 1D array with large elements is used to wrap around the neck region of a subject. In comparison, in configuration 210, a 1D array with relatively small elements is used to wrap around the neck region of the subject. As illustrated, configuration 200 may create more coverage of the neck region. However, the offset distance between the 1D array coil and the neck region is relatively large (e.g., 5-15 cm or even more) from the most concave part of the neck, giving arise to reduced SNR. In contrast, configuration 210 has smaller coil elements that allow for a smaller offset distance to the neck, improving the SNR at the neck. Indeed, when a 1D planar array with large uniform elements is wrapped cylindrically around the neck, vertical (superior-inferior) coverage is adequate but a relatively large spatial offset will result. But when smaller uniform elements are used instead, the spatial offset is reduced albeit with reduced superior-inferior coverage. If the small elements are combined as a two-dimensional array, as illustrated by configuration 220, the same dilemma will persist as in configuration 200 because the array cannot easily bend in the vertical dimension while wrapped in a cylinder.

To address the dilemma, configuration 230 introduces a hybrid array that demonstrates how the outer rows bend out to create a “cone” when wrapped around the neck as a cylinder. In some cases, the coil elements are arrayed on a flexible substrate (for example, a fabric). In some embodiments the coils are permanently affixed to the underlying substrate, The two outer rows of the array of coil elements are arranged on fixed curves. As illustrated, when the array is wrapped around the neck region, the upper outer row towards the head (superior) and the lower outer row towards the shoulder (inferior) are bent into cone shapes to conform to these curvatures. Although the illustrated example shows one row of coil elements, either the upper outer row or the lower outer row can have more than one row of coil elements. Additionally or alternatively, some embodiments can include the upper outer row(s) but without the lower outer row(s), or the lower outer row(s) but without the upper outer row(s). In the meantime, the center row, when bent, can provide adequate superior-inferior coverage over the neck segment and with sufficient proximity to the skin. As illustrated, the center row of coil elements, when resting in an unbent state, are arranged on a straight line, for example, on the flexible substrate laid flat. The center row may not be limited to one row only. In various examples, more than one row can form the central portion of the array configuration. The advantages of the illustrated array with the curved rows include the ability to maintain optimum overlap between the rows of coils, despite the number of (or varied size of) additional coil elements to achieve the needed curvature. As coil development is typically an iterative process requiring modifications in coil number and layout, this approach can make this iterative process much more efficient.

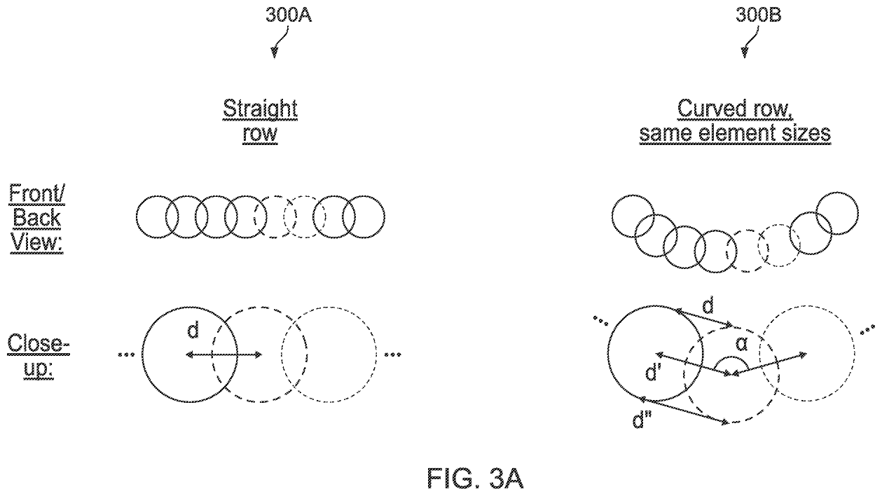

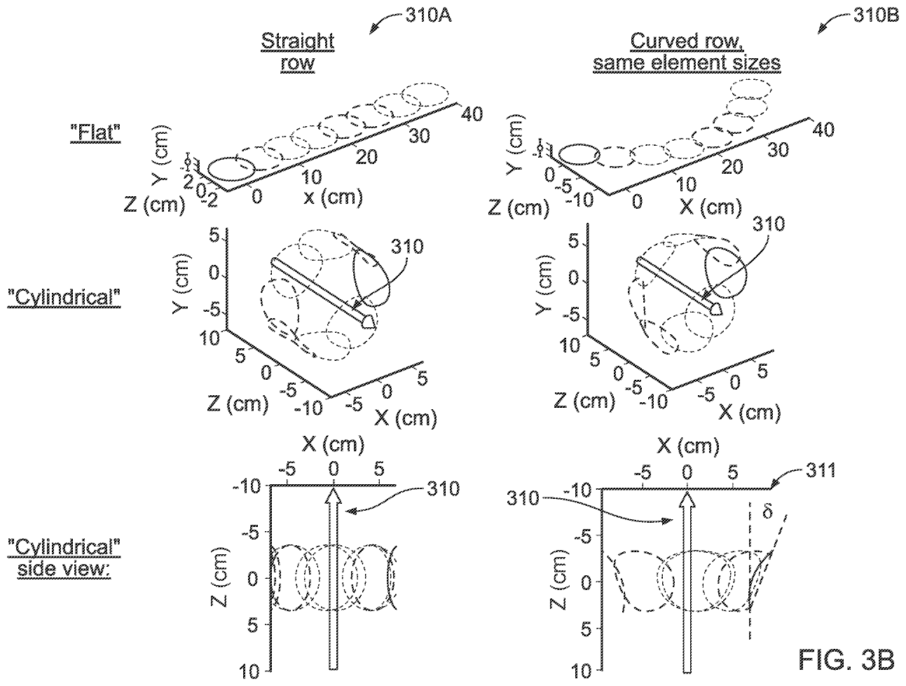

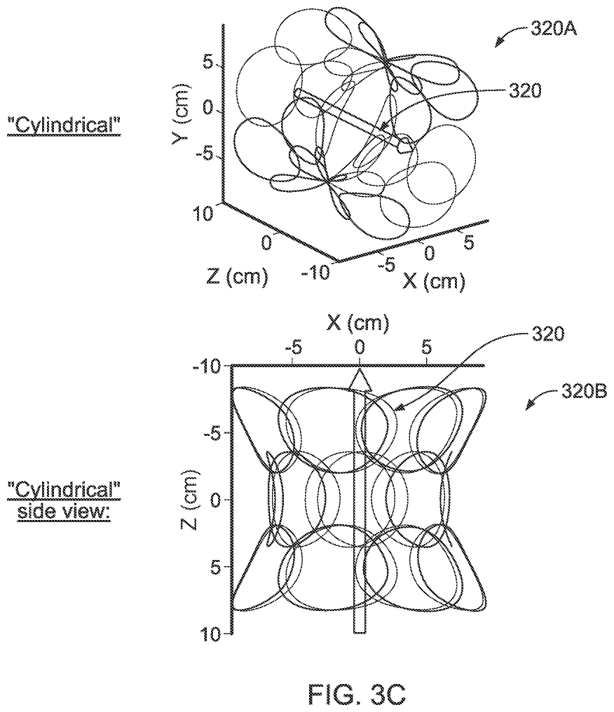

Referring to FIGS. 3A to 3C, two approaches can allow the array to conform to curved surfaces. To allow for arrays to conform to a curvature when bent, the first approach is to include one or more rows of elements with at least one curved row of elements. FIG. 3A illustrates a comparison of a straight row of elements with a curved row of elements. Here, the straight row 300A, and a curved row 300B are both 1D arrays. Each array has elements with identical sizes. The element sizes in both arrays are the same. A close-up of the front-view of the arrays shows the arrangement of three of the circular elements, indicated with different lines. The variable, d, represents the distance between adjacent elements in the same row on the concave side of the curved row, while d′ represents the distance between the center of adjacent elements in the curved row. The variable d″ represents the distance between adjacent elements on the convex side of the curved row. In order for the curved row to flare out when wrapped around, a non-180 degrees obtuse angle, α, is needed such that d′>d.

When a straight row 300A is bent, e.g., to form an axial cylinder, each element will be parallel to the axis of the cylinder. In this case of a cylindrical coverage, the diameter of the cylinder (D) as a function of the interspacing (d) between each element for a row of N elements is:

$$D = \frac{d}{sin ( \frac{π}{N} )} \qquad ( 1 )$$

This relationship can be approximated to D=Nd/π when N is a large number (for example, 32, 64, or even larger). For a curved row 300B that flares out, when the curved row 300B is bent and wrapped around like a cylinder, the curved row 300B forms a cone instead. In order for the narrower part of the curve to match the straight row, one can compute an increased spacing (d′) and a non-180 degree obtuse angle between the lines connecting the centers of adjacent elements (α) that is d′=d+2r cos(α/2), where r is the radius or width of the element (assuming the element is a perfect circle). The diameter at the center of the curved row (D′) is D′=Nd′/π. Therefore, for a desired conical angle to the same cylindrical axis (δ) one can simply derive that as a function of:

$$δ = a sin ( \frac{N}{π} cos ( α / 2 ) ) \qquad ( 2 )$$

FIG. 3B shows examples of three-dimensional (3D) views of the straight row array and the curved row array. The 3D view of the straight row is shown in column 310A while the 3D view of the curved row is shown in column 310B. Here, the straight row is initially laid flat and all elements are aligned in a straight line (hence the name straight row). When the straight row is bent and wrapped around a cylinder, a cylindrical coverage is created (in which an axis of each element is substantially orthogonal to the axis of the cylinder). In comparison, when the curved row is laid out, the coil elements are spread in a curve. When the curved row is bent and wrapped around a cylinder, the curved row forms a desired conical angle as shown in FIG. 3B. The example thus highlights a “flare-out” cone, where the narrower diameter part of the cone is matched to that of a straight row of coil elements. As illustrated, arrows 310 in the cylinder views point to the longitudinal axis of the cylinder. The curved row produces a cone with conical angle 311, δ, relative to the axis 310 of the cylinder. The dashed lines in the “cylindrical” side view, applying a curved row, are projections of the vertical axis and the “cone” slope surface, drawn to illustrate the conical angle.

Alternatively or additionally, rather than to utilize similarly-sized elements as the straight row, the second approach uses a curved row that includes different sized elements as long as d′ is defined as d′=d+2r cos(α/2). Specifically, for the flare-out cone, it is likely that elements larger than the adjacent straight row would be utilized. Also, for other geometries, the cone can “flare in” instead of out. Specifically, the wider diameter of the cone in such cases are matched to the straight row of coils instead. In such cases, d′=d−2r cos(α/2) and the equation for δ will still apply. For a flare-in cone, smaller size elements may be used instead for the curved row.

FIG. 3C demonstrates two views of an example of an array that combines rows of curved arrays. In this example, a total of three rows includes a straight row located between two curved rows. As illustrated in the cylindrical view 320A and the cylindrical side view, the array includes one straight row between two curved rows. The example shows identical size elements, with a conical angle of 25 degrees in both curved rows. This conical angle is the angle of δ, relative to the axis (320) of the cylinder.

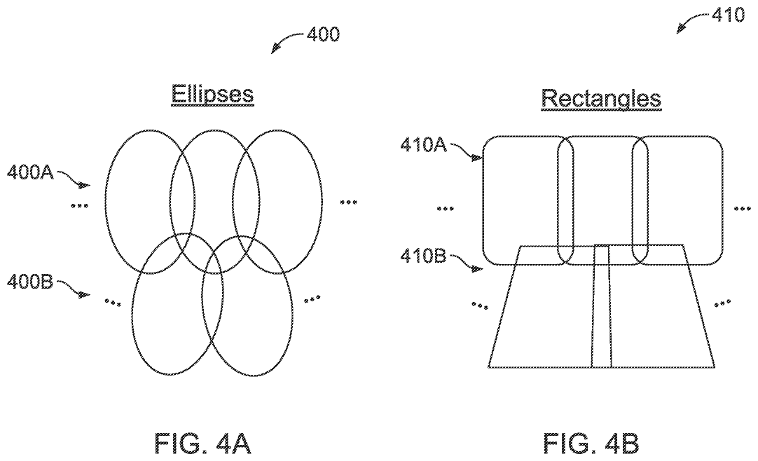

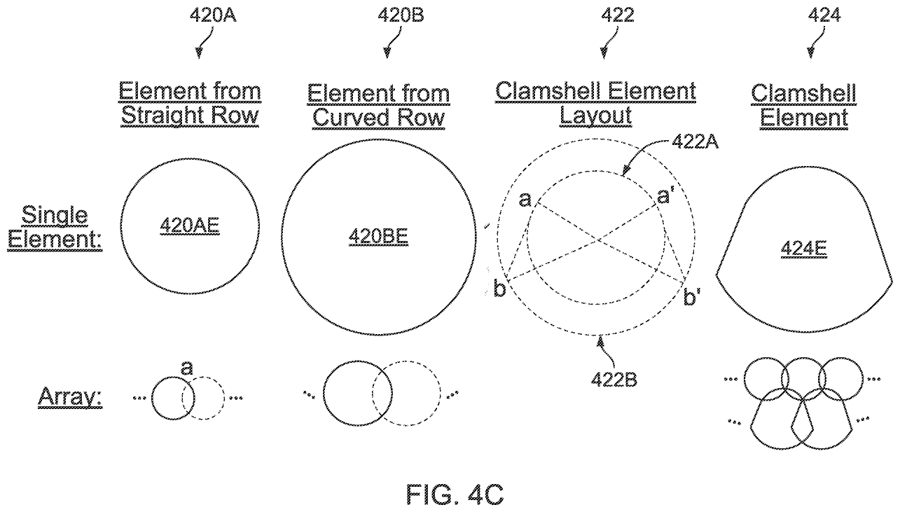

Additionally or alternatively, implementations may combine curved rows of elements of various shapes, rather than a circular shape. FIG. 4A shows a configuration 400 which includes a straight array and a curve row, both including array elements in elliptical shape. FIG. 4B also shows a configuration 410 in which the straight array includes rectangular-shaped elements while the curved row includes elements in a trapezoidal shape. Without loss of generality, the same design applies these non-circular shapes (or any closed loop topology). In the case of ellipses, the equations for circles can be adapted for the major/minor radii of the ellipse. In the case of rectangles, the curved row may include utilize trapezoids, whereby the ratio between the top and base of the trapezoid can be proportional to the ratio of the diameters of the cylinder, D and D′.

Alternatively or additionally, implementations may include a circular “clamshell” element that is circumscribed partially by two circles with radii of differing dimensions. The purpose of the clamshell element is to retain one radius for the row of elements belonging to the clamshell, while allowing the other radius to match an element from a separate row. Examples of use cases can include imaging of the neck, where the diameter of the head and shoulders are larger than the neck, leading to larger elements at the head and shoulders for deeper coil sensitivity profiles.

FIG. 4C shows an example of a clamshell element layout 422 and an array configuration 424 that includes a row of clamshell elements according to some implementations. By way of illustration, in this example, the array row incorporating the clamshell element 424E has a larger radius than the straight array row that is on top of the array row. In this illustration, the clamshell element 424E can be designed by aligning the centers of both circular elements 420AE and 420BE. As illustrated by clamshell element layout 422, a point denoted, “a,” is the intersection between adjacent elements 420AE on the straight array row 420A (that is, a coil element from straight row 420A). The clamshell element 424E is left-right symmetric, with a similar point, “a′,” denoted on the other side of the lateral span. The points “b” and “b′” are likewise denoted as the intersections from the lines tangential to “a” and “a′,” respectively, with the element 420BE from the curved row 420B (that is, a coil element from straight row 420B). The clamshell 424E is circumscribed on one side (top) by the inner circle 422A between points “a” and “a′,” and on the other side (bottom) by the outer circle 422B between points “b” and “b′,” as well as the lines “ab” and “a′b′”. In doing so, the extent of the overlap between the clamshell row and the straight row matches that of the straight row above it, and the gap between both rows are also matched by coils with the same dimensions (e.g., radii).

In some cases, to draw projection lines from points “a” and “a′” to “b” and “b′,” the outer radius may not exceed the inner radius by more than approximately 70%, assuming that a 25% overlap is applied (which can be typical for phased-array coils). In addition, the “straight row” can also be replaced by another curved row of regular circular elements, or a curved row of clamshell elements. In addition, the elements in the straight row may be larger than the curved clamshell elements. As such, points “b” and “b′” are then the intersection points between the larger elements, and points “a” and “a′” are points at which their respective tangents intersect with points “b” and “b′”.

As demonstrated herein, coil arrays can be constructed to include various coils with a variety of shapes and sizes, while maintaining a conformal shape to accommodate the desired curved, concave geometries of, for example, a target anatomy such as the head/neck/chest wall/shoulder region, the axillary (armpit) region, the pelvic/inner thigh region, elbow, wrist, fingers, knee, ankle, and toes. Indeed, implementations of the present disclosure describe effective and systematic methodologies of designing and constructing coil arrays for curved and concave surfaces.

As used herein, the terms “comprises” and “comprising” are to be construed as being inclusive and open-ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

At least portions of the implementations of the subject matter and the functional operations described in this specification can be implemented in digital electronic circuitry, in tangibly-implemented computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible non-transitory program carrier for execution by, or to control the operation of, data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparati, devices, and machines for processing data, including, by way of example, a programmable processor, a computer, or multiple processors or computers. The apparatus can also be or further include special purpose logic circuitry, e.g., a central processing unit (CPU), a FPGA (field programmable gate array), or an ASIC (application specific integrated circuit). In some implementations, the data processing apparatus and/or special purpose logic circuitry may be hardware-based and/or software-based. The apparatus can optionally include code that creates an execution environment for computer programs, e.g., code that constitutes processor firmware, a protocol stack, a database management system, an operating system, or a combination of one or more of them. The present disclosure contemplates the use of data processing apparatuses with or without conventional operating systems, for example Linux, UNIX, Windows, Mac OS, Android, iOS or any other suitable conventional operating system.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a communication network. While portions of the programs illustrated in the various figures are shown as individual modules that implement the various features and functionality through various objects, methods, or other processes, the programs may instead include a number of sub-modules, third party services, components, libraries, and such, as appropriate. Conversely, the features and functionality of various components can be combined into single components as appropriate.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., a central processing unit (CPU), a FPGA (field programmable gate array), or an ASIC (application specific integrated circuit).

Computers suitable for the execution of a computer program include, by way of example, can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media (transitory or non-transitory, as appropriate) suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The memory may store various objects or data, including caches, classes, frameworks, applications, backup data, jobs, web pages, web page templates, database tables, repositories storing business and/or dynamic information, and any other appropriate information including any parameters, variables, algorithms, instructions, rules, constraints, or references thereto. Additionally, the memory may include any other appropriate data, such as logs, policies, security or access data, reporting files, as well as others. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube), LCD (liquid crystal display), or plasma monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

The term “graphical user interface,” or GUI, may be used in the singular or the plural to describe one or more graphical user interfaces and each of the displays of a particular graphical user interface. Therefore, a GUI may represent any graphical user interface, including but not limited to, a web browser, a touch screen, or a command line interface (CLI) that processes information and efficiently presents the information results to the user. In general, a GUI may include a plurality of user interface (UI) elements, some or all associated with a web browser, such as interactive fields, pull-down lists, and buttons operable by the business suite user. These and other UI elements may be related to or represent the functions of the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN), a wide area network (WAN), e.g., the Internet, and a wireless local area network (WLAN).

The computing system can include clients and servers. A client and server are generally remote from each other and typically interact through a communication network. The relationship of client and server arises by virtue of computer programs running on the respective computers and having a client-server relationship to each other.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

What is claimed is:

1. A phased-array coil assembly comprising:
a first row of coil elements arranged along a first curve on a flexible substrate when the flexible substrate is flat,
wherein the first row of coil elements are configured to conform to a first curved body region in a first cone shape such that when the flexible substrate is bent and wrapped, the first row forms the first cone shape to cover the first curved body region with sufficient proximity to cause an increased sensitivity to magnetic resonance (MR) signals from the first curved body region,
wherein the first cone shape is determined, at least in part, by the first curve.

2. The phased-array coil assembly of claim 1, further comprising:
a second row of coil elements arranged along a straight line on the flexible substrate when the substrate is flat,
wherein the second row of coil elements are configured to conform to a second curved body region in a cylinder shape such that when the substrate is bent and wrapped, the second row forms the cylinder shape to surround the second curved body region with sufficient proximity to cause an increased sensitivity to MR signals from the second curved body region, and
wherein each coil element in the first row is sized to have a first area and each coil element in the second row is sized to have a second area.

3. The phased-array coil assembly of claim 2, wherein the first area is larger than the second area.

4. The phased-array coil assembly of claim 2, wherein the first area is identical to the second area.

5. The phased-array coil assembly of claim 2, wherein the first area is smaller than the second area.

6. The phased-array coil assembly of claim 2, further comprising:
a third row of coil elements arranged along a second curve on the flexible substrate when the flexible substrate is flat,
wherein the third row of coil elements are configured to conform to a third curved body region in a second cone shape such that when substrate is bent and wrapped, the third row forms the second cone shape to cover the third curved body region with sufficient proximity to cause an increased sensitivity to MR signals from the third curved body region, and
wherein the second cone shape is determined, at least in part, by the second curve.

7. The phased-array coil assembly of claim 6,
wherein the first curved body region corresponds to an area between a head region and a neck region,
wherein the second curved body region corresponds to the neck region, and
wherein the third curved body region corresponds to an area between the neck region and a shoulder region.

8. The phased-array coil assembly of claim 6,
wherein the first curved body region corresponds to an area between a groin region and a hip region,
wherein the second curved body region corresponds to the groin region, and
wherein the third curved body region corresponds to an area between the groin region and a thigh region.

9. The phased-array coil assembly of claim 2,
wherein the first row and the second row are neighboring rows.

10. The phased-array coil assembly of claim 9,
wherein each coil element of the first row overlaps with one or more coil elements of the second row, and
wherein each coil element of the second row overlaps with one or more coil elements of the first row.

11. The phased-array coil assembly of claim 6, wherein the second row and the third row are neighboring rows.

12. The phased-array coil assembly of claim 11,
wherein each coil element of the third row overlaps with one or more coil elements of the second row, and
wherein each coil element of the second row overlaps with one or more coil elements of the third row.

13. The phased-array coil assembly of claim 6,
wherein each element in the third row is sized to have a third area, and
wherein the third area is larger than the second area.

14. The phased-array coil assembly of claim 6,
wherein a coil element in the third row is shaped in one of: a circular shape, an elliptical shape, a trapezoidal shape, or a clamshell shape.

15. The phased-array coil assembly of claim 1, wherein a coil element in the first row is shaped in one of: a circular shape, an elliptical shape, a trapezoidal shape, or a clamshell shape.

16. The phased-array coil assembly of claim 2, wherein a coil element in the second row is shaped in one of: a circular shape, an elliptical shape, a rectangular shape.

17. A magnetic resonance imaging (MRI) scanner, comprising:
a main magnet configured to generate a volume of magnetic field with field inhomogeneity below a defined threshold, the main magnet including a bore area sized to accommodate at least a body part of a subject;
a phased-array coil assembly capable of wrapping around the body part, the phased-array coil assembly comprising:
a first row of coil elements arranged along a first curve on a flexible substrate when the flexible substrate is flat, wherein the first row of coil elements are configured to conform to a first curved body region in a first cone shape such that when the flexible substrate is bent and wrapped around the first curved body region, the first row forms the first cone shape to cover the first curved body region with sufficient proximity to cause an increased sensitivity to MR signals from the first curved body region, wherein the first cone shape is determined, at least in part, by the first curve, and wherein the body part includes the first curved body region;
gradient coils configured to generate gradient pulses that provide perturbations to the volume of magnetic field such that MRI signals encoding an MRI image according to encoding information from the gradient pulses are emitted from the body part and are subsequently acquired by the coil assembly wrapped around the body part; and
a control unit in communication with the gradient coils and the coil assembly and configured to operate: (i) the gradient coils to generate the gradient pulses and (ii) the coil assembly to acquire MRI signals emitted from the body part that encode the MRI image.

18. The MRI scanner of claim 17, wherein the phased-array coil assembly further comprises:
a second row of coil elements arranged along a straight line on the flexible substrate when the flexible substrate is flat,
wherein the second row of coil elements are configured to conform to a second curved body region in a cylinder shape such that when the flexible substrate is bent and wrapped, the second row forms the cylinder shape to surround the second curved body region with sufficient proximity to cause an increased sensitivity to MR signals from the second curved body region,
wherein the body part includes the second curved body region, and
wherein each coil element in the first row is sized to have a first area and each coil element in the second row is sized to have a second area.

19. The MRI scanner of claim 18, wherein the phased-array coil assembly further comprises:
a third row of coil elements arranged along a second curve on the flexible substrate when the flexible substrate is flat,
wherein the third row of coil elements are configured to conform to a third curved body region in a second cone shape such that when the flexible substrate is bent and wrapped, the third row forms the second cone shape to cover the third curved body region with sufficient proximity to cause an increased sensitivity to MR signals from the third curved body region, and
wherein the body part includes the third curved body region.

20. The MRI scanner of claim 19,
wherein the first curved body region corresponds to an area between a head region and a neck region,
wherein the second curved body region corresponds to the neck region, and
wherein the third curved body region corresponds to an area between the neck region and a shoulder region.

21. The MRI scanner of claim 19,
wherein the first curved body region corresponds to an area between a groin region and a hip region,
wherein the second curved body region corresponds to the groin region, and
wherein the third curved body region corresponds to an area between the groin region and a thigh region.
